(19) United States
(12) Patent Application Publication
(10) Pub. No.: US 2014/0264031 A1
(43) Pub. Date: Sep. 18, 2014

(54) TRACE GAS DETECTION SYSTEM

(71) Applicant: IMRA AMERICA, INC., Ann Arbor, MI (US)

(72) Inventors: Martin E. FERMANN, Dexter, MI (US); Kevin F. Lee, Ann Arbor, MI (US); Andrew A. Mills, Ann Arbor, MI (US)

(73) Assignee: IMRA AMERICA, INC., Ann Arbor, MI (US)

(21) Appl. No.: 14/178,985

(22) Filed: Feb. 12, 2014

Related U.S. Application Data
(60) Provisional application No. 61/793,913, filed on Mar. 15, 2013.

(51) Int. Cl. G01J 3/42; A61B 5/00; G01J 3/10

(52) U.S. Cl. CPC G01J 3/42; G01J 3/108; A61B 5/0075
USPC 250/339.02; 250/339.08; 250/339.07

(57) ABSTRACT

The present invention relates to a trace gas detection system. At least one embodiment includes a frequency spectrum comprising a 1st comb and an enhancement cavity characterized by having a 2^{nd} comb of spectral resonances. The enhancement cavity contains a sample gas for spectroscopic measurement. A dither mechanism is configured to modulate the relative spectral position between the combs at a dither frequency, f_{d}. The dither mechanism, in conjunction with a feedback mechanism, stabilizes the location of said 1^{st} comb lines with respect to the resonances of said 2^{nd} comb over a time scale much greater than a dither period, T_{d}=1/f_{d}. A time-averaged output from the enhancement cavity is provided to a spectroscopic measurement tool, for example a Fourier transform spectrometer. The system is capable of detecting volatile organic compounds, endogenous compounds, and may be configured for cancer detection.

### CROSS REFERENCE TO RELATED APPLICATIONS

This application is based on and claims priority under 35 USC 119(e) from U.S. Provisional Application No. 61/771,346 filed Mar. 1, 2013, the contents of which are incorporated herein by reference in their entirety

### FIELD OF THE INVENTION

The present invention relates to trace gas detection system based on frequency combs.

### BACKGROUND

In recent years interest in high resolution optical spectroscopy has increased. The following exemplary patents, published patent applications, and publications relate to light sources for precision optical frequency measurement and applications of the same in high resolution spectroscopy:

Holzwarth et al., U.S. Pat. No. 6,724,788 entitled ‘Method and device for generating radiation with stabilized frequency’;

Holzwarth et al., U.S. Pat. No. 6,785,303, entitled ‘Generation of stabilized, ultra-short light pulses and the use thereof for synthesizing optical frequencies’;

Haensch et al., U.S. Pat. No. 6,897,959, entitled “Frequency comb analysis”;

Fermann et al., U.S. Pat. No. 7,190,705, entitled ‘Pulsed laser sources’;

Fermann et al., U.S. Pat. No. 7,649,915, entitled ‘Pulsed laser sources’;

Hartl et al., U.S. Pat. No. 7,809,222, entitled ‘Laser based frequency standards and their applications’;

Gohle et al., U.S. Pat. No. 8,120,773, entitled ‘Method and device for cavity enhanced optical vernier spectroscopy’;

Fermann et al., U.S. Pat. No. 8,120,778: entitled ‘Optical scanning and imaging systems based on dual pulsed laser systems’;

Giaccari et al. U.S. Patent Application Pub. No. 2011/0043815, entitled ‘Referencing of the Beating Spectra of Frequency Combs’;

Vodopyanov et al., U.S. Patent Application Pub. No. 2011/0058248, entitled ‘Infrared frequency comb methods, arrangements and applications’;

T. Sizer, ‘Increase in laser repetition rate by spectral selection’, IEEE J. Quantum Electronics, vol. 25, pp. 97-103 (1989);

S. Diddams et al., ‘Molecular fingerprinting with the resolved modes of a femtosecond laser frequency comb’, Nature, vol. 445, pp. 627 (2007);

R. Gebs et al., ‘1 GHz repetition rate femtosecond OPO with stabilized offset between signal and idler frequency combs’, Opt. Expr., vol. 16, pp. 5397-5405 (2008)

F. Adler et al., Phase-stabilized, 1.5 W frequency comb at 2.8 μm-4.8 μm, Opt. Lett., vol. 34, pp. 1330-1332 (2009),

A. Foltynowicz et al., ‘Optical frequency comb spectroscopy’, Faraday Discussions, vol. 150, pp. 23-31, 2011

Kohlhaas et al., ‘Robust laser frequency stabilization by serrodyne modulation’, Opt. Lett., vol. 37, pp. 1005 (2012); and

N. Leindecker et al., Opt. Expr., ‘Octave-spanning ultrafast OPO with 2.6-6.1 μm instantaneous bandwidth pumped by femtosecond Tm-fiber laser’, Opt. Expr., 20, 7046 (2012).

Advances in frequency measurement methods and systems have occurred over the past several years with the use of optical frequency combs. However, high resolution, broadband measurement in the mid-IR spectral region and beyond remains challenging.

Cavity enhanced spectroscopy systems are difficult to implement in a reliable fashion due to the complexity of the required electronics and components required to facilitate stable coupling of a source to the cavity. To reduce the complexity of the electronics required to couple a frequency comb laser into a cavity, and to reduce amplitude noise from cavity length fluctuations, a dither lock to the cavity can be implemented. Dither locking of enhancement cavities to modelocked lasers is well known in the state of the art and was for example described in T. Gherman and D. Romanini, ‘Modelocked Cavity—Enhanced Absorption Spectroscopy’, Opt. Express, vol. 10, 1033 (2002). In some configurations, when implementing dither locking, the comb spacing of the frequency ruler and the enhancement cavity are adjusted to be integer multiples of each other. The relative location of the cavity or source comb modes is then scanned in frequency space by about one free spectral range (FSR) of the cavity, though smaller and larger scan ranges can also be implemented. As the cavity length is swept, the resonant frequencies of the cavity change as well, such that every comb line will be coupled into the cavity for some small period of time. In the absence of dispersion, all comb lines would be coupled in at the same point in time. The presence of the sample gas in the cavity and other intracavity components may introduce dispersion. With dispersion, a slight mismatch between the cavity mode spacing and the frequency ruler means that different comb lines will couple to the cavity at slightly different times. With this kind of coupling, the average cavity transmission is reduced significantly compared to a system configuration where the cavity is locked to the frequency ruler. For a dither scan range of 10 MHz and a cavity line width of 10 kHz, the average cavity transmission can be reduced by up to a factor on the order of 1000. Therefore, dither-locked cavity enhanced spectroscopy requires relatively high laser powers and has not been demonstrated due to the lack of appropriate laser sources in the mid-IR.

### SUMMARY

Frequency combs comprise a highly developed technology platform that has been used in many advanced optical technologies. Here we present a system configuration based on frequency combs that can be used for cavity enhanced and cavity enhanced direct comb spectroscopy.

At least one embodiment of a trace gas detection system includes an optical source which produces as a primary output a frequency spectrum having a 1st comb with a 1^{st} comb spacing within a 1^{st} spectral range. An enhancement cavity, containing a sample gas for spectroscopic measurement, is configured to receive the primary output of the optical source and to produce a secondary output. The enhancement cavity may be characterized by having a 2^{nd} comb of approximately equidistant spectral resonances and a 2^{nd} comb spacing within a 2^{nd} spectral range. The first 1^{st} spectral range and 2^{nd} spectral range overlap. The system further includes a dither mechanism configured to modulate the relative position between the 1^{st} comb and the 2^{nd} comb at a dither frequency, f_{d}, and to impart variations of the relative position in optical frequency space larger than an optical linewidth of the cavity resonances. A feedback mechanism is coupled to the dither mechanism to stabilize the location of the 1^{st} comb lines with respect to the resonances of the 2^{nd} comb on a time scale much greater than a dither period, T_{d}=1/f_{d}. A Fourier transform spectrometer is configured to receive the secondary output, and to measure the spectrum of a time-averaged signal transmitted by the cavity over a time scale much longer than T_{d}.

At least one embodiment of a trace gas detection system includes an optical source producing as a primary output a frequency spectrum having a 1st comb with a 1^{st} comb spacing within a 1^{st} spectral range. The first spectral range includes wavelengths>1600 nm. An enhancement cavity, containing a sample gas for spectroscopic measurement, is configured to receive the primary output of the optical source and to produce a secondary output. The enhancement cavity may be characterized by having a 2^{nd} comb of approximately equidistant spectral resonances and a 2^{nd} comb spacing within a 2^{nd} spectral range. The 1^{st} spectral range and 2^{nd} spectral range overlap. The system further includes a dither mechanism configured to modulate the relative position between the 1^{st} comb and the 2^{nd} comb at a dither frequency, f_{d}, and to impart variations of the relative position in optical frequency space larger than an optical linewidth of the cavity resonances. A feedback mechanism is coupled to the dither mechanism to stabilize the location of the 1^{st} comb lines with respect to the resonances of the 2^{nd} comb on a time scale much greater than a dither period, T_{d}=1/f_{d}. A spectroscopic measurement tool, which includes an optical detection system, is arranged for frequency resolved detection of a time-averaged signal transmitted through the enhancement cavity.

At least one embodiment of a trace gas detection system includes an optical source producing as a primary output a frequency spectrum having a 1st comb with a 1^{st} comb spacing within a 1^{st} spectral range. An enhancement cavity, containing a sample gas for spectroscopic measurement, is configured to receive the primary output of the optical source and to produce a secondary output. The enhancement cavity may be characterized by having a 2^{nd} comb of approximately equidistant spectral resonances and a 2^{nd} comb spacing within a 2^{nd} spectral range. The 1^{st} spectral range and 2^{nd} spectral ranges overlap. The system further includes a dither mechanism configured to modulate the relative position between the 1^{st} comb and the 2^{nd} comb at a dither frequency, f_{d}, and to impart variations of the relative position in optical frequency space larger than an optical linewidth of the cavity resonances. A spectroscopic measurement tool is configured to receive the secondary output, and to measure the spectrum of a time-averaged signal transmitted by the cavity over a time scale much longer than T_{d}=1/f_{d}. The spectroscopic tool is arranged to provide a signal for synchronization of dithering with spectroscopic data acquisition.

Any form of frequency comb can be implemented. For example, frequency combs based on quantum cascade lasers, micro-resonators, or mode locked lasers can be used. Mode locked lasers based on fiber, semiconductor or solid-state technology can be implemented. Appropriate amplification stages can further be used for signal amplification.

To shift the spectral output of the modelocked lasers into a spectral region of interest, a frequency shifting device such as supercontinuum generator, difference frequency generator, optical parametric oscillator (OPO), or optical parametric amplifier (OPA) can be used.

To couple the light from a frequency comb system into an enhancement cavity a dither lock is implemented, where the comb modes of either the cavity or the frequency comb system are rapidly dithered around an average value.

The comb modes can be dithered using a modulation of the carrier envelope offset frequency of the frequency comb, its comb mode spacing or the cavity length of the enhancement cavity. Additional optical frequency shifter(s) can also be incorporated between the comb source and the enhancement cavity.

To facilitate spectroscopic measurements the enhancement cavity is filled with a gas and the spectrum transmitted through the cavity is detected using dispersive optical systems such as diffraction gratings or VIPAs and one or two dimensional detector arrays.

Alternatively, spectral detection can be performed with conventional Fourier transform spectrometers.

In order to minimize amplitude fluctuations when using a Fourier transform spectrometer, it is beneficial to synchronize the zero-crossings in the Fourier transform detection system with the dither function of the enhancement cavity.

The spectroscopy system as discussed here can be used for trace gas detection such as that used in medical breath analysis. Of particular interest is the detection of molecules and volatile organic compounds (VOC) with absorption bands in the 3-5 μm and the 5-12 μm spectral ranges, with endogenous compounds being of particular interest.

### BRIEF DESCRIPTION OF THE DRAWINGS

FIG. 1A schematically illustrates a conventional frequency comb.

FIG. 1B illustrates an embodiment of a polarization maintaining fiber oscillator suitable for use in various embodiments, which allows for phase control of the oscillator.

FIGS. 1C-1E illustrates some of the possible approaches for control of carrier envelope offset frequencies associated with a frequency comb system.

FIG. 2 schematically illustrates a difference frequency generator (DFG) based frequency comb.

FIG. 3 schematically illustrates a mid IR source based on an optical parametric generator.

FIG. 3a schematically illustrates an output spectrum of an OPO.

FIG. 4 schematically illustrates an example of a trace gas detection system in which the output of a frequency comb source or frequency ruler is utilized in combination with an enhancement cavity and Fourier transform spectrometer (FTS) for cavity enhanced spectroscopy. The arrangement employs various control mechanisms for monitoring and stabilization of the comb and cavity, including a frequency dither mechanism to lock the ruler or comb frequencies to the enhancement cavity.

FIG. 4A schematically illustrates a system for cavity enhanced spectroscopy using cavity length dithering.

FIG. 4B schematically illustrates a system for cavity enhanced spectroscopy using frequency comb dithering.

FIG. 4C schematically illustrates a system for cavity enhanced spectroscopy using both frequency comb dithering and cavity length dithering.

FIG. 4D schematically illustrates a system for cavity enhanced spectroscopy using dithering in conjunction with a Fourier transform spectrometer.

FIG. 5 schematically illustrates an example showing temporal evolution of modulation and clock signals when using cavity dithering with a Fourier transform spectrometer.

FIG. 6 schematically illustrates a gas delivery system as used in conjunction with cavity enhanced comb spectroscopy according to an embodiment of the present invention.

FIG. 7 schematically illustrates a breath analysis system suitable for medical applications according to an embodiment of the present invention.

### DETAILED DESCRIPTION

Optical spectroscopy has experienced a great resurgence in interest since the introduction of optical frequency combs as, for example, exemplified in U.S. Pat. No. 6,785,303: ‘Generation of stabilized, ultra-short light pulses and the use thereof for synthesizing optical frequencies’ and U.S. Pat. No. 6,724,788: ‘Method and device for generating radiation with stabilized frequency’. Frequency combs are disclosed in '303 as having an output spectrum which can be written as fₙ=nfᵣₑₚ+f₀ (e.g.: column 2, lines 4-11), where n is an integer and fₙ denotes the frequencies of individual comb modes. The frequency spectrum 100 of such a conventional frequency comb laser is further illustrated in FIG. 1. To first order the frequency spectrum is determined by fᵣₑₚ and f₀. The frequency comb can be generated with any suitable comb source, which may include micro-resonators, quantum cascade lasers (QCL), or a mode locked laser and fᵣₑₚ corresponds to the comb spacing or the repetition rate of the pulses generated with a mode locked laser, whereas f₀ corresponds to the carrier envelope offset frequency. In some embodiments dual combs may be provided in which each comb is generated by one or a combination of the above sources. In '303, f₀ is also referred to as the slip frequency (e.g.: column 2, line 8), which is the same for all comb modes in such devices. For a good frequency comb source, ideally the phases between individual comb modes are fixed in time with respect to each other and vary only slowly across the pulse spectrum due to effects such as intra-cavity dispersion, thermal effects or power fluctuations. Opto-mechanical transducers in conjunction with electronic feedback loops may be used to set or stabilize fᵣₑₚ or f₀ in such comb systems, as also disclosed in U.S. Pat. No. 7,809,222, ('222), entitled ‘Laser based frequency standards and their applications’, and U.S. Pat. No. 7,649,915, ('915), entitled ‘Pulsed laser sources’. U.S. Pat. Nos. 7,809,222 and 7,649,915 are hereby incorporated by reference in their entirety.

Some frequency comb systems utilize mode-locked fiber oscillators to produce an output spectrum as illustrated in FIG. 1. FIGS. 1A-1E illustrate frequency comb generation and various techniques to set or stabilize fᵣₑₚ or f₀ in such comb systems. FIG. 1B, which is reproduced from the '915 patent, illustrates several components of an exemplary mode-locked fiber oscillator. The oscillator 101 includes a saturable absorber module 120, and collimation and focusing lenses 121 and 122, respectively. The saturable absorber module 120 further comprises a saturable absorber 123 which acts as a highly reflective (HR) cavity mirror that is preferably mounted onto a first piezo-electric transducer 124. The first piezo-electric transducer 124 can be modulated to control, for example, the repetition rate of the oscillator 101. The oscillator 101 further comprises an oscillator fiber 125 that is preferably coiled onto a second piezo-electric transducer 126. The second piezo-electric transducer 126 can be modulated for repetition rate control of the oscillator 101. The oscillator fiber 125 is preferably polarization-maintaining and has a positive dispersion although the designs should not be so limited. The dispersion of the oscillator cavity can be compensated by a fiber grating 127 which preferably has a negative dispersion and is also used for output coupling (OC). It will be understood that a positive dispersion fiber grating and a negative dispersion cavity fiber may also be implemented. Furthermore, the fiber grating 127 can be polarization-maintaining or non-polarization-maintaining.

The pump light for the oscillator 801 can be directed via a polarization-maintaining wavelength division multiplexing coupler 128 from a coupler arm 129 attached to a preferably single-mode pump diode 130. The pump current to the pump diode 830 can be modulated to stabilize the beat signal frequency and the carrier envelope offset frequency using feedback based on the signal at one selected frequency.

Monitoring and control of f₀ and fᵣₑₚ provide for full characterization of the comb. The oscillator output, which may be amplified with an optional fiber amplifier, may be supplied to an f-2f interferometer (not shown) in which the well-known self-referencing technique can be used to extract f₀ via detection of a beat signal. The repetition rate fᵣₑₚ may be monitored or stabilized in an arrangement having an electronic phase locked loop comprising high speed photodetector(s), RF-amplifier(s), RF bandpass filter(s), phase detector(s) and loop filters, as discussed in '222.

Electronic feedback loops may be used stabilize the comb. In particular, FIGS. 1C-1E of the present application (also disclosed in '915) illustrate some of the possible approaches for controlling the beat signal related to the carrier envelope offset frequencies associated with the system of FIGS. 1A. FIGS. 1C-1D illustrate some of the approaches to using the beat signal frequency to control the repetition rate as well as the carrier envelope offset frequency. As shown in FIG. 1C, a pump current 140 can be changed, wherein a change in the pump current can cause a change of the beat signal frequency and more particularly the carrier envelope offset frequency. This dependence can be used to phase lock the beat signal frequency to an external clock in a similar way as a voltage-controlled oscillator in a traditional phase locked loop can be used.

As shown in FIGS. 1D and 1E, the absolute position of f₀ can be controlled by adjusting the temperature of the fiber grating 127 with a heating element 142. Alternatively, pressure applied to the fiber grating 127 can also be used to set f₀ using, for example, a piezo-electric transducer 144. Since the pressure applied to the fiber grating 127 can be modulated very rapidly, modulating the pressure on the grating 127 can also be used for phase locking f₀ to an external clock.

Many possibilities exist. Further information regarding the above arrangements may be found in '915 and '222. In accordance with the present invention such arrangements may be utilized in various embodiments or modified in various ways for use in high resolution spectroscopy systems, as will be further discussed below. For example, as will become apparent in the present disclosure, control circuits for rapidly modulating pump current supplied to the oscillator, grating pressure, and/or grating temperature may be advantageously used to vary f₀, and such rapid modulation may be over a small modulation depth compared to the operating range of the device.

For any instrumentation applications, frequency combs based on mode-locked fiber lasers have several advantages over both mode-locked bulk solid state lasers and mode-locked diode lasers. Mode-locked fiber lasers offer typically superior noise properties compared to mode-locked diode lasers and can be packaged in smaller spaces than mode-locked bulk solid state lasers. Mode-locked fiber lasers can be produced with excellent thermal and mechanical stability. In particular, passively mode-locked fiber lasers can be constructed with few and inexpensive optical components, suitable for mass production, as disclosed in U.S. Pat. No. 7,190,705 ('705) and U.S. Pat. No. 7,809,222 ('222). The dispersion compensated fiber lasers as disclosed in '705 provide for the construction of low noise frequency comb sources. Also disclosed were designs of fiber lasers operating at repetition rates in excess of 1 GHz. As a compact alternative to mode locked fiber lasers, frequency combs based on micro-resonators or quantum cascade lasers can also be used.

Low-noise operation of fiber lasers limits timing jitter, allowing optimized control of the timing of the pulses. The '705 patent disclosed the first low noise fiber-based frequency comb source. Low noise operation was obtained by controlling the fiber cavity dispersion in a certain well-defined range. Low noise operation of fiber frequency comb sources reduces the noise of the carrier envelope offset frequency f₀ of the laser to a negligible level, and also facilitates measurement and control of f₀.

Exemplary applications of optical frequency combs have been demonstrated in Fourier transform spectroscopy based on two frequency comb lasers operating at slightly different repetition rates as discussed in U.S. Patent Application Pub. No. 2011/0043815, entitled ‘Referencing of the Beating Spectra of Frequency Combs’, Other spectroscopy applications include measuring the response function of samples with frequency combs as discussed in ‘Frequency comb analysis’, U.S. Pat. No. 6,897,959. Many other examples can be found in the literature.

In addition to the construction of frequency combs as discussed in '303, other implementations of frequency combs have been demonstrated. One such implementation of a frequency comb is shown in FIG. 2. Here a nonlinear device in the DFG stage acts as a difference frequency generator (DFG) and produces a frequency comb from a pulse train originating from a mode locked pump laser source. With a DFG the carrier envelope offset frequency f₀ is also fixed across the whole output spectrum, and with a DFG, f₀=0.

Other nonlinear optical devices have been demonstrated where the relation fₙ=nfᵣₑₚ+f₀ also holds. Examples of such optical devices are highly nonlinear optical fibers that generate a supercontinuum output, for example as described in U.S. Pat. No. 7,809,222. Another example can be a degenerate synchronously pumped optical parametric oscillator (DOPO), for example as described in ‘Infrared frequency comb methods, arrangements and applications’, U.S. Patent Application Pub. No. 2011/0058248, and in N. Leindecker et al., Opt. Expr., ‘Octave-spanning ultrafast OPO with 2.6-6.1 μm instantaneous bandwidth pumped by femtosecond Tm-fiber laser’, Opt. Expr., 20, 7046 (2012). Because the DOPO is synchronously pumped, its repetition rate is the same as the repetition rate of the pump laser.

In some other devices, the relation fₙ=nfᵣₑₚ+f₀ does not hold for the output frequency range of the device. An example of such a device is a non-degenerate OPO (NOPO), where generally the idler and signal frequency have different unstable carrier envelope offset frequencies f₀ᵢ and f₀ₛ respectively, even when the pump f₀ₚ is stabilized. As described in F. Adler et al., ‘Phase-stabilized, 1.5 W frequency comb at 2.8 μm-4.8 μm’, Opt. Lett., vol. 34, pp. 1330-1332 (2009), additional electronic feedback loops need to be implemented inside the NOPO that stabilize the carrier envelope offset frequencies of either the signal or idler frequency, f₀ₛ or f₀ᵢ respectively. If f₀ₚ is stabilized, and either f₀ₛ or f₀ᵢ is also stabilized, the carrier envelope offset frequencies at both signal and idler frequencies can be determined because, due to energy conservation, f₀ₚ=f₀ₛ+f₀ᵢ.

In other devices, such as weakly non-degenerate OPOs (WOPOs), the difference of f₀ₛ and f₀ᵢ can also be stabilized by taking advantage of overlapping signal and idler spectra, as described in R. Gebs et al., ‘1 GHz repetition rate femtosecond OPO with stabilized offset between signal and idler frequency combs’, Opt. Expr., vol. 16, pp. 5397-5405 (2008).

In yet other devices, degenerate doubly resonant synchronously pumped OPOs (DOPOs) were suggested as versatile mid IR sources for operation with stable carrier phase when pumped with a fiber laser comb source, see N. Leindecker et al., Opt. Expr., ‘Octave-spanning ultrafast OPO with 2.6-6.1 μm instantaneous bandwidth pumped by femtosecond Tm-fiber laser’, Opt. Expr., 20, 7046 (2012).

In yet other devices, synchronously pumped non-degenerate optical parametric oscillator (DNOPOs) were suggested as versatile mid IR sources for operation with stable carrier phase in U.S. Patent Application No. 61/764,355, ('355), entitled “Optical frequency ruler”, filed Feb. 13, 2013, which is hereby incorporated by reference in its entirety. An example of a typical optical arrangement for a DNOPO is shown in FIG. 3. A DNOPO configuration is particularly attractive because it lowers the pump power that is required to initiate parametric oscillation inside the cavity compared to WOPOs and NOPOs. Low pump powers are preferred for operation of OPOs at high repetition rates (300 MHz and higher) with relatively low pump power lasers. Such DNOPOs are particularly useful sources for the mid-IR spectral range. Notably, for any OPOs, the expression fₙ=nfᵣₑₚ+f₀ for the output of the device does not hold across the output spectrum, because the carrier envelope offset frequencies of signal and idler can be different in general. However, as shown in '355, the carrier envelope offset frequency of the signal and idler inside the OPO can be readily stabilized and determined with high precision.

FIG. 3 schematically illustrates a DNOPO 300 according to an embodiment. The arrangement includes a DNOPO cavity and a pump laser. As used in the present application, and particularly when referring to DNOPOs or other parametric systems, the phrases pump laser, pump laser source, pump source or similar expressions related to the pump arrangement are not to be construed as necessarily limited to only an oscillator. Thus, a pump laser may be an oscillator, but may also include downstream optical amplifier(s) to increase the energy of pump pulses to a suitable level. The pump laser can include a mode locked fiber laser, however, suitable mode locked solid-state or semiconductor lasers can also be implemented. In the example of FIG. 3 the pump laser generates short picosecond (ps) or femtosecond (fs) pulses at a constant repetition rate and with sufficient power to pump the DNOPO, for example a few hundreds of mW, as will be discussed below. Such DNOPOs were disclosed in '355 and are not further discussed here.

A generic frequency ruler generated by such a DNOPO is shown in FIG. 3a. The DNOPO is represented by a nonlinear optical system and may include signal processor(s) (not shown) to monitor and stabilize the comb lines of the pump laser and DNOPO. The pump in this example produces a comb spectrum 405 given by fₚ=pfₚ+f₀ₚ. As discussed in '355, in contradistinction to a conventional frequency comb, the output of the nonlinear optical system contains a frequency shifted ruler comprising at least two distinct spectral sections with respective comb spectra fₙ=nfₙ+f₀₁ and fₘ=mfₘ+f₀₂. A residual pump spectrum can also be contained in the output. For the example of the DNOPO, the comb modes with subscript n refer to the idler spectrum 310-a, whereas the comb modes with subscript m refer to the signal spectrum 310-b. The signal and idler spectra do not need to be adjacent to each other; in general the carrier envelope offset frequencies for signal and idler will be different, i.e. there is a change in the carrier envelope offset frequency between signal and idler or there is at least one discontinuity in the frequency spacing of the comb modes when going from the signal to the idler part of the output spectrum. Thus, as a function of frequency, the carrier envelope offset frequency (CEOF) is not constant but exhibits a discontinuity in the transition between the signal and idler spectrum The output can also comprise additional spurious signals arising from nonlinear frequency mixing between pump, signal and idler.

Examples of optical sources for spectroscopy applications, and more particularly for embodiments directed to cavity enhanced spectroscopy, include: frequency combs, mode locked lasers, DFG, OPOs, OPAs and frequency shifted mode locked lasers based on, for example, supercontinuum generation.

Various embodiments of frequency comb lasers can be constructed at comb spacings of >300 MHz, or preferably >500 MHz and most preferably at comb spacings>1 GHz for applications in direct comb spectroscopy. Methods for direct comb spectroscopy were for example disclosed in U.S. patent application Ser. No. 12/955,759, ('759), entitled: ‘Frequency comb source with large comb spacing’, filed Nov. 29, 2010. In brief, when using a frequency ruler with a comb spacing>500 MHz, bulk optic components can be readily used to resolve individual comb lines and the individual comb lines can then be detected with a detector array. One such implementation was discussed in '759.

A scheme with a solid-state laser based multi-GHz repetition rate comb system and a two dimensional angular dispersion element as well as a two dimensional detector array was previously described in S. Diddams et al., ‘Molecular fingerprinting with the resolved modes of a femtosecond laser frequency comb’, Nature, vol. 445, pp. 627 (2007). However, a system with a fiber laser pumped GHz-level repetition rate OPO was not considered. With advancements as described herein, low noise OPO frequency rulers at repetition rates of 1 GHz and higher can be constructed which make such schemes very attractive.

A frequency resolution equivalent to the ruler line width can be obtained by slowly scanning the comb spacing or carrier envelope offset frequency of the frequency ruler, and detecting with a resolution approximately twice higher than the repetition rate of the frequency ruler, sufficient to separate individual comb lines. Integrating the signal over adjacent frequencies that are identified as belonging to a comb line gives the signal at that comb line. This results in a frequency resolution that is several orders of magnitude better than the frequency resolution of the detection system with a standard light source.

For example, a typical Fourier transform spectrometer (FTS) can have a resolution of 500 MHz, which is sufficient to resolve comb lines for comb spacings of 1 GHz. Using a Fourier transform spectrometer provides a significant cost benefit from using a single channel detector rather than a two dimensional detector array, and Fourier transform spectrometers can have very broad bandwidths, up to the entire bandwidth of the detector. For example, HgCdTe detector provides for detection over an optical bandwidth from about 2 to 13 μm.

For comb spacings larger than around 10 GHz, individual comb lines can, for example, be resolved using two or more conventional diffraction gratings in series or multiple passes or reflections from a single grating. A grating system has the cost advantage of a single-dimensional detector array rather than a two-dimensional array. Compared to a Fourier transform spectrometer, it can have a faster acquisition rate, and does not have moving parts, but it has the disadvantage of a much lower detection bandwidth.

Large comb spacings further allow the implementation of broadband differential absorption spectroscopy. In such a system, the position of the comb lines can be slowly scanned and at the same time modulated at high frequencies in frequency space in order to generate a time dependent modulation of the signal impinging on the comb resolved detection system. Such schemes are well known from single laser spectroscopy. Many other spectroscopic techniques can be adapted to broadband detection where the principle requirement is the optical resolution of individual comb lines.

A particularly attractive scheme for broadband trace gas detection is based on cavity enhanced trace gas detection as disclosed in U.S. Patent Application No. 61/617,482, ('482), entitled ‘Methods for precision optical frequency synthesis and molecular detection’, filed Mar. 29, 2012, to Fermann et al. The '482 application is hereby incorporated by reference in its entirety. When combined with a frequency ruler or frequency comb with large frequency spacing, broadband detection of multiple gas species can be performed simultaneously.

As described in patent and non-patent literature a frequency comb source has sometimes been associated with arrangements in which one or both of the repeitition rate, fᵣₑₚ, or carrier envelope offest, fₒ, are phased locked to reference signals, for example, with phase locked loop(s). It is to be understood that such phase locking is not necessary to the practice of each and every embodiment of the present invention. For example, unless other specified, carrier envelope offest, fₒ, may slip or be free-floating with allowable variation (which may be pre-determined). A frequency comb source (or frequency ruler) may operate in the absence of phase locking. In some embodiments one or both of the repetition rate, fᵣₑₚ, or carrier envelope offest, fₒ, are phased locked to reference signals, and may be preferred for certain high-resolution spectroscopy applications.

FIG. 4 schematically illustrates an arrangement 400 for cavity enhanced spectroscopy in accordance with an embodiment of the present invention. A frequency ruler 410, or more generally an optical source, may include a mode locked oscillator together with optional components for stabilization of f₀ and/or fᵣₑₚ as discussed above with respect to FIGS. 1 and 1B-1E. In some embodiments a frequency comb and DFG may be utilized as discussed with respect to FIG. 2. As yet another alternative, a DNOPO arrangement may be utilized as discussed with respect to FIG. 3. It is to be understood that the arrangements are not mutually exclusive and can be combined in various ways to meet specific application requirements. Other possible optical sources include, for example, a QCL comb source, a general frequency comb source, or more generally a frequency ruler, may be characterized as having as a primary output a first comb, or more generally, a first ruler 450 which includes equidistant optical frequencies within a first spectral range, and with a 1^{st} comb spacing.

The output of the frequency ruler (e.g.: a frequency comb) is coupled to an enhancement cavity 420. The enhancement cavity may contain a gas sample for spectroscopic measurement. The enhancement cavity may be characterized by having a comb of approximately equidistant spectral reonances 460-a within a second spectral range (as determined by the coatings of the cavity mirrors). The second spectral range is to overlap the first spectral range associated with the frequency comb output 450.

In certain embodiments a frequency comb source 410 may produce a comb spacing in the range from about 50 MHz to greater than 1 GHz. Enhancement cavity 420 may be configured such that a linewidth of a resonance is in the range from about 1 kHz to 100 kHz. In some embodiments the enhancement cavity may have a comb spacing (e.g.: second comb spacing) which is an integer multiple of the first comb spacing, or an integer fraction of the first comb spacing.

The arrangement 400 employs various mechanisms for monitoring and stabilizing at least the frequency ruler 410 and cavity 420. A frequency dither mechanism is included to lock the ruler or comb frequencies (1^{st} comb) to the resonances (2^{nd} comb) of the enhancement cavity. Thus, with comb 450 as input to enhancement cavity 420, the output of the enhancement cavity will include a second frequency comb corresponding to a secondary output 460 with the comb lines 460-b spaced at the approximately equidistant spectral resonances 460-a and centered, on the average, at the peaks of the enhancement cavity resonances 460-a. The spectrum of the time-averaged signal transmitted by enhancement cavity 420 is an output available for downstream spectroscopic measurement as will be further discussed below.

A control unit 440 as schematically represented in FIG. 4 is in communication with at least ruler 410, cavity 420, and can be further interfaced to the Fourier transform spectrometer 430. In at least one embodiment control unit 440 is arranged to receive optical signals and information obtained from the ruler 410, cavity 420, and FTS 430 and to provide control signals to system components. It is to be understood that control unit 440 and associated feedback mechanisms, sensors, and other components may be arranged with any suitable combination of components distributed throughout the system, lumped into a single system controller, or arranged with dedicated, local control circuitry and components associated with ruler 410, cavity 420, and/or FTS 430. Control unit 440 may comprise a system computer, including a FTS system computer, and provide ability to communicate with external devices over a communications link using communication protocols common to computer communication, such as RS-232, TCP/IP, CAN etc.

In particular, a frequency dither mechanism is included which modulates the relative position between the first comb produced by the frequency comb source 450 and the second comb of spectral resonances 460-a. The modulation occurs at a dither frequency, f_{d}, and a corresponding dither period, T_{d}. A dither frequency may be in the range from about 100 Hz to about 100 kHz, and in some embodiments may be at or near 10 kHz. A feedback mechanism, which may include one or more servo loops for monitoring/controlling the comb source and/or enhancement cavity, is arranged to center, on the average, comb lines of comb 450 within cavity resonances 460-a of the enhancement cavity 420.

The resulting output 460 of the enhancement cavity, as illustrated in FIG. 4 shows the centered comb lines 460-b (solid) centered, on the average, on cavity resonances 460-a (dashed lines). The time scale for providing time-averaged signals is much longer than the dither period T_{d} (inverse of the dither frequency). It can be seen from FIG. 4 that the secondary output from the enhancement cavity 420 includes comb lines 460-a having a comb spacing which corresponds to the comb spacing of the enhancement cavity 420. The spectrum of the time-averaged signal transmitted by the cavity, over several dither cycles and with a time scale much longer than T_{d}, is then available for spectroscopic measurement.

The system further includes a tool for spectroscopic measurement, for example Fourier transform spectrometer (FTS) 430 which provides as an output the spectrum 470 of a sample. In a conventional FTS arrangement a time delay is introduced between two arms of an interferometer. The time delay between the arms can be varied by translating a reflector. The recombined light intensity is then detected and recorded as a function of path delay, which is measured, for example, by a HeNe reference laser which simultaneously propagates through the interferometer. In accordance with the present invention the spectrometer is advantageously arranged for operation with the frequency comb and corresponding optical pulses by synchronizing FTS data acquisition to the dithering via control unit 440 and feedback mechanisms as associated therewith, as discussed above. Other aspects, features, and advantages of various embodiments and arrangements will become more apparent from the following examples, discussion, and the accompanying drawings.

A system configuration implementing a frequency ruler dither locked to an enhancement cavity for cavity enhanced spectroscopy in the mid IR spectral region is shown in FIG. 4A. The output of the frequency ruler or frequency comb system is coupled into an enhancement cavity bounded by at least 2 high reflectivity mirrors using appropriate mode-matching optics (not shown). The cavity further contains at least one mirror mounted onto a piezo-electric transducer that enables a fast modulation of the cavity length. Additional translation stages may also be included to enable a slow modulation of the cavity length. A gas delivery system is further included (not shown) to deliver a sample in the gas phase to the cavity. The light transmitted by the cavity is further detected with an optical detection system (D1). An additional detection system (D2) is further incorporated to enable spectroscopic measurements, and may be included as part of the FTS 430 arrangement. In various embodiments D2 can, for example, comprise a single detector, a Fourier transform spectrometer or a one or two dimensional detector array. Additional optical filters can also be incorporated. D1 may receive light that is reflected from the entrance of the enhancement cavity (421-a), light that has passed through the cavity (421-b) or, depending on the type of spectrometer, D2 may serve the function of D1. The light may be directed from the enhancement cavity to D1 and D2 via appropriate optical filters, beam splitters and mirrors which are not separately shown.

The round trip time of the cavity is further locked to the frequency comb spacing of the cavity with a dither lock and the servo loop which may be included in or interfaced to control unit 440. An appropriate electronic locking scheme for implementing a dither lock was for example described with respect to FIG. 8 of M. J. Thorpe et al., ‘Cavity-enhanced direct frequency comb spectroscopy’, Appl. Phys. B., vol. B91, pp. 397-414 (2008). In brief, the cavity is dithered across the resonance using a triangle waveform applied to the piezo-electric transducer (PZT) that controls the cavity length. Since the cavity drifts slowly, an additional DC offset is applied to the PZT. This DC offset is regulated such that the cavity dither is always approximately centered around the resonant length of the input light.

An electronic control scheme generally referred to as a flip flop servo loop may be utilized and is well known in the state of the art. The feedback circuit is implemented as follows: From the triangle scan waveform applied to the PZT, an auxiliary square wave SQW1 is generated which flips at each change of the scan direction. A photodetector-comparator combination generates a second square wave SQW2 with rising edges aligned to the points where the cavity transmission reaches a pre-set threshold from below. The threshold may be set at about 3-10 times the peak-peak noise level so that a stable square wave is obtained. This signal is used as the clock on a D-flip-flop, sampling SQW1 applied to the D-input of the flip-flop at the rising edges of SQW2. The mark-to space ratio of the D-flip-flop's output wave SQW3 is now a measure of the alignment of the cavity resonance to the triangle dither scan. That is, if the transmission threshold is reached exactly at the center of the scan, the mark-to-space ratio would be 1:1. An integrator converts the mark-to-space ratio of SQW3 to a proportional DC voltage which is used for slow feedback control of the PZT offset voltage. The above description is to serve only as an example and many alternative implementation of flip flop servo loops or similar arrangements may be used in the servo loop of FIG. 4A.

A dither scan range of one free spectral range ensures that the frequency comb will be coupled into the cavity at some point during the sweep. This scan range also means that most of the time, light will not be coupled into the cavity. To increase cavity transmission, the dither scan range can be reduced to a fraction of the free spectral range of the cavity. The flip flop circuit described above can be implemented to keep the dither centered on resonance, however, other electronic control loops may also be used for the same purpose. In the case of a flip-flop circuit, the light reflected from or transmitted through the cavity can be sampled with detector D1 to keep the cavity dither on resonance. The dither frequency and the dither magnitude are easily controlled by the frequency and magnitude applied to the drive signal of the intra-cavity PZT. The useful dither frequency is limited by the finesse of the cavity. The cavity must be resonant for enough time for the intracavity field to become large enough to enable strong coupling into the cavity.

Moreover, the cavity spacing can be adjusted depending on which spectral region is being detected; this accounts for a mismatch in mode spacing between the frequency ruler and the enhancement cavity due to dispersion.

An alternative to dithering the cavity length is to dither the laser frequencies as shown in FIG. 4B. By scanning the comb spacing, or the carrier envelope offset frequency, the laser comb lines are made to oscillate around the cavity transmission resonances. In this example additional slow control loops to control the length of the cavity can also be incorporated. A frequency ruler 410 is used as the signal source and an enhancement cavity 420, preferably with a gas supply system, is incorporated. Two detectors are used to enable cavity length locking via a servo loop and spectroscopic detection, respectively.

In an exemplary implementation, a DNOPO can be implemented as a frequency ruler. The comb spacing of the DNOPO can be dithered by dithering the cavity length of the DNOPO pump laser (e.g.: the frequency ruler or comb source). Alternatively, or in combination, the carrier envelope offset frequency of a DNOPO can be changed by changing the carrier envelope offset frequency or repetition rate of the DNOPO pump laser or the DNOPO cavity length. Controlling the laser comb source 410 rather than the cavity 420 has the advantage that the laser frequency combs can be controlled at a much faster rate than the speed of moving an enhancement cavity mirror. For example, the carrier envelope offset frequency of a mode locked laser is often controlled by adjusting the power of its pump laser, which can be done quickly by combining the main pump with a faster supplementary pump.

For example, referring back to FIGS. 1B and 1C, the pump light supplied to the oscillator 101 from the pump diode laser 130 may be rapidly varied over a limited modulation depth to generate supplemental pump current. In some embodiments a second laser diode (not shown) may be modulated at a rapid rate, and the beam from the second (supplemental) diode is combined with the pump diode beam with bulk optic(s) or a fiber combiner in such a way as to provide a pump beam with a single spatial mode to the oscillator (not shown). Still faster methods can be used, such as using a graphene modulator as in Lee et al. Opt. Lett. 37, 3084 (2012) to control the carrier envelope offset frequency of a laser cavity at MHz speeds. Use of a graphene modulator or, alternatively a Mach-Zehnder or other integrated modulator can avoid imparting a wavelength chirp associated with diode laser current modulation.

There are also many methods of controlling the repetition rate, for example, using piezoelectric transducers to move a laser cavity mirror, or to stretch a spool of optical fiber, thereby controlling the cavity length as, for example, illustrated and discussed with respect to FIG. 1B in which piezoelectric transducers 124, 126 are shown for control of the HR cavity mirror and coiled oscillator fiber 125, respectively.

The frequency comb lines can further be modulated using external modulators. For example an acousto-optic frequency shifter (AOFS in FIG. 4B) can be used to control the mid infrared frequency comb directly by adding or subtracting frequencies on the order of 10-100 MHz. Such an AOFS can for example be implemented in front of an enhancement cavity as shown in FIG. 4B or alternatively, an AOFS can be inserted in front of an optical parametric oscillator, DFG or OPA stage. For example when inserting the AOFS in front of the signal or idler arm of a DFG stage, a non-zero carrier envelope offset frequency can be obtained even when the signal and idler arm are derived from the same signal source. Such schemes are well known in the state of the art and not further shown here.

Using an AOFS is particularly beneficial because it separates the dithering function from the control of the frequency comb laser, The faster response time enabled by such comb dithering can be used to reduce the dither range, increasing transmission through the enhancement cavity. The faster response time of comb dithering can also be used to lock the comb laser to the cavity, yielding less amplitude noise than when locking the cavity to the comb laser. For this method to provide single comb-line resolution the cavity length must fluctuate enough so that all frequencies are occasionally transmitted through the cavity.

For some applications, the frequency dependent beam pointing from the AOFS may be a limitation. However, as discussed in the '482 application, this can be eliminated by double passing the AOFS as described, for example, in E. A. Donley et al., ‘Double-pass acousto-optic modulator system’, Rev. of Scientific Instruments, vol. 76, pp. 063112 (2005). A double-pass through an AOFS effectively doubles the modulation frequency, therefore the AOFS drive frequency needs to be divided by two to produce the right frequency correction to the cw laser.

Frequency dithering and cavity length scanning can be combined to yield a high-throughput, high-resolution system, with relatively simple locking requirements, as shown in FIG. 4C. Again, the cavity components may comprise the same elements as discussed with respect to FIGS. 4A and 4B. In this implementation, the cavity length can be swept over more than one, or several (e.g.: 5, 10, 20) free spectral ranges, for example at 1 kHz, ensuring transmission of all frequencies at regular intervals. The laser frequency can be quickly dithered around the slowly changing cavity resonance at a much higher rate, for example 100 kHz, using, for example, the same dithering methods described above, providing high transmission for all frequencies

A low cost spectroscopic detection system for cavity-enhanced spectroscopy is a Fourier transform spectrometer (FTS), which can provide high resolution, and broad bandwidth. A standard FTS includes an interferometer where the time delay between the two arms can be scanned by a moving carriage (e.g.: translation stage) with a reflector. The recombined light intensity is detected and recorded as a function of path delay, which is measured by, for example, a HeNe reference laser which simultaneously propagates through the interferometer. However, a conventional FTS operates with continuous, or effectively continuous light, rather than the intermittent, time-separated pulses that result from dithered transmission through a cavity.

In at least one embodiment of the present invention the system 400 is arranged for use with dither-controlled cavity enhanced detection schemes by synchronizing the FTS data acquisition to the dithering based on a control signal. The FTS 430 may be configured to sample the signal transmitted through the enhancement cavity 420 in synchronism with zero crossings of an interference signal generated with the FTS internal reference laser (not shown). A dither period, T_{d}, may also be derived from the zero crossings and used to control a dither mechanism coupled to optical source 410 and/or cavity 420 via the feedback mechanism. In one implementation, the FTS detector is coupled with a long time constant, for example about 1 msec. As such, the pulses arriving at the dither rate appear effectively continuous for the detection. The carriage speed is then synchronized to the dither rate to have the same number of bursts of light for each acquired point. For example, if one data point is acquired for every FTS reference-laser wavelength of path delay, and a group of pulses arrive at a group rate of 1 kHz, the carriage speed will be an integer fraction of (reference-laser wavelength*1 kHz). By synchronizing the carriage speed to the dither rate, shot noise problems from acquiring irregular numbers of pulses per data point are avoided.

In a related implementation, as shown in FIG. 4D, the dither frequency can be directly derived from the FTS carriage movement and a reference laser. In this example an optical path delay timing signal 430-a is derived from the FTS. The dither frequency is then applied to the enhancement cavity length as shown, or to the frequency comb. In some embodiments, comb laser control is preferred because the frequency comb can be controlled at a faster rate than the enhancement cavity length. Reference lasers are generally used to track the irregular movement of the moving carriages in conventional Fourier transform spectrometers (FTS). As the carriage is scanned, the reference laser interferences (from the two arms of the FTS interferometer) produce a nearly regular sinusoidal oscillation. This oscillation can then be used as the clock for the dither frequency, automatically matching the carriage and dither speed. In FIG. 4D, the same components as described with respect to FIGS. 4A-4C may be used.

An example of timing signals for synchronizing the FTS, dither, and acquisition are illustrated in FIG. 5. The FTS produces an offset sinusoid from the reference laser interferences (reference laser, top curve). After filtering to center the sinusoid around 0 volts, the zero crossings can generate a square wave clock (clock, 2^{nd} from top). Integrating the clock yields a triangle wave (dither, 3^{rd} from top) appropriate for dithering, for example, with use of a mirror with a piezoelectric transducer. An additional slow servo loop as, for example, included with the feedback mechanism, stabilizes the cavity length of the enhancement cavity, providing uniform or other desired intervals between transmission peaks through the cavity light, (bottom curve). In some embodiments the FTS may be configured to sample more than two cavity transmission peaks between two zero crossings, i.e. a uniform number of transmission peaks between zero crossings. Samples may be obtained at time intervals much smaller that the intervals between adjacent zero crossings. In at least one embodiment the dither period, T_{d}, as exemplified by the triangle wave, is derived from the zero crossings of the reference laser interference pattern. An acquisition trigger (trigger, 2^{nd} from bottom) for the FTS detectors is derived from the zero crossings of the dither signal, since the cavity transmission occurs at the center of the dither. Many variations are possible, and may be similar to the conventional practices of synchronizing the FTS acquisition to the zero crossings of the reference laser interferogram.

In at least one embodiment, a more flexible implementation is provided. Synchronization of the reference laser zero crossings, dither and signal acquisition, is replaced by relatively fast data acquisition, at a rate higher than the dither rate, such that the burst of light for each dither period is well-resolved. The multiple peaks within a burst due to the dispersion do not need to be resolved. For example, all signals are acquired simultaneously at 1 MHz, while the dither rates are on the scale of 10 kHz. Two signals are acquired for the two quadratures of the reference laser, and the two signals at both interferometer outputs are measured. The reference laser provides the path delay for each measurement. Acquiring both quadratures provides the absolute path delay for each measurement, as is common in FTS. Acquiring both interferometer outputs has the advantage of reducing noise by taking the difference of the two intensities, as is common for FTS. The sum of both outputs also provides a measurement of the cavity transmission, providing the function of detection system D1. In at least one embodiment, at least 1 MHz detection may be implemented using commercially available data converters and associated digital processing hardware. Higher data acquisition rates are feasible, for example operation in the range from about 1 MHz to 50 MHz in embodiments for very high speed operation.

In this implementation, most data points have low light levels, and correspond to non-resonant cavity lengths. These can be ignored, thereby reducing noise. Data points identified as corresponding to resonant cavity transmission, for example by thresholding the sum of the two interferometer outputs, are kept and used in calculating the spectrum. If the acquisition rate is fast enough that a transmission burst lasts for more than one data point, the intensities can be summed and treated as a single data point, with the requirement that the carriage speed is slow enough that the burst is complete before the path delay has changed by the desired minimum path delay interval, for example, a reference laser wavelength. In the case where absolute position is measured, acquired data points can be averaged over multiple scans. Beyond the usual benefit of scan averaging on noise, there is the additional benefit that the data points at different positions do not need to be taken in succession, but can come from combining many scans. The localization of a burst to within a path delay interval is still required, and this can be achieved by faster dithering of, for example, the laser frequency.

An exemplary design of a gas delivery system appropriate for use with enhancement cavities is further shown in FIG. 6. In the illustrated implementation, a carrier gas, such as nitrogen, helium, or argon, is continuously flowed over the sample material (solid or liquid), carrying a small amount of sample into and out of the cavity vessel. The sample can be heated to increase the vapor pressure of the sample. Alternately a gaseous sample or a calibration gas can be introduced directly. A purge gas such as nitrogen can be used to clean the chamber between measurements. The pressure within the cavity is preferably on the order of one atmosphere in order to reduce contamination from air, but lower pressures can be used to decrease the pressure broadening of molecules, increasing the sensitivity and the ability to distinguish between various molecules. For example supersonic expansion of gases into the cavity (as well known in the state of the art) can also be implemented. In cases where the available sample is too limited for a continuous flow, a static gas fill can be used, with the additional requirements that the cavity will need to be constructed to higher vacuum standards, or the cavity will need to be placed inside another system that is either in vacuum, or more simply, filled with nitrogen. The acquisition time will then be limited by the contamination rate.

In some configurations, the system can be configured to measure the concentration of volatile organic compounds (VOCs), with endogenous compounds being or particular interest. Some VOC's of particular interest include acetaldehyde, acetone, benzene, toluene, ethylbenzene, formaldehyde, decane, dodecane, undecane, 1,2,4-trimethylbenzene, hexanal and isopropanol. Spectroscopy has an inherent advantage over mass spectrometry in that it can discriminate between molecules with the same nominal mass to charge ratio (m/z), for example ethane and formaldehyde (m/z=30), methanol/methylamine (32), nitrogen dioxide/dimethylamine (47), acetone/isobutane/butane (58), carbonyl sulfide/isopropanol (60), dimethylsulfide, trimethyl amine/ethanethiol (62), carbondisulfide/propanethiol/isopropanethiol (76), hexene/methyl cyclopentane (84), and propylbenzene/1,2,4-trimethyl benzene (120). These molecules with the same nominal mass can often be differentiated with high-resolving-power mass spectrometers, which come with increased time, cost, and complexity, such as dual MS followed by collisions as in MS/MS or by selected ion flow-tube mass spectrometry (SIFT). Dual MS, MS/MS as well as SIFT are well known in the state of the art and not further explained here. Such techniques require complicated analysis of the fragmentation patterns of a single selected ion, which further increases the analysis time, and wastes sample. Sensitivity is expected in the ppmv or ppbv range, where ppmv or ppmb stands for parts per million or billion volume fraction in air.

To further simplify a broadband trace gas detection system, the enhancement cavity can further be substituted with a multi-pass gas cell such as a Herriott or White cell as well known in the state of the art.
Trace gas detection systems as described here can be readily implemented for medical breath analysis using well known gas delivery systems for transporting breath samples to an appropriate enhancement cavity or a multi-pass cell as illustrated in FIG. 7. Ideally, breath samples are measured by directly breathing into the cavity. A tedlar bag, or other inert container, can be used as a buffer to control the flow of breath into the cavity. A breath sample can also be acquired offsite in a tedlar bag, and transported to the cavity for analysis. In various embodiments of the present invention IR detection capability from about 1.6 μm up to about 15 μm is provided, suitable for spectral measurement in 3-6 μm and the 5-15 μm spectral ranges. Any molecules with absorption bands in the spectral region from 2-15 μm can so be detected with a very high sensitivity for example ammonia, isotopic CO2, ethene, methylamine, dimethylamine, and trimethylamine. Of particular interest is the detection of molecules and volatile organic compounds (VOC) with absorption bands in the 3-5 μm spectral ranges, such as methane (CH₄), ammonia (NH₃), ethane (C₂H₆), ethene (C₂H₄), propane (C₃H₈), formaldehyde (CH₂O), nitric oxide (NO), hydrogen sulfide (H₂S), ethanol (CH₃CH₂OH), ozone (O₃), acetone (CH₃OCH₃), carbonyl sulfide (COS), sulfur dioxide (SO₂), benzene (C₆H₆), methanol (CH₃OH), isobutane ((CH₃)₃CH), isopropanol (CH₃CHOHCH₃), dimethylsulfide (CH₃SCH₃), isoprene (CH₂C(CH₃)CHCH₂), pentane (CH₃CH₂CH₂CH₂CH₃), toluene (C₆H₅CH₃), butane (CH₃CH₂CH₂CH₃), 1-hexene (CH₂CHCH₂CH₂CH₂CH₃), methyl nitrate (CH₃NO₃), pyridine (C₅H₅N), octane (C₈H₁₈), 2-hexene (CH₃CHCHCH₂CH₂CH₃), 3-hexene (CH₃CH₂CHCHCH₂CH₃), methyl cyclopentane (c-C₅H₉—CH₃), methanethiol (CH₃SH), ethanethiol (CH₃CH₂SH), 1-propanethiol (CH₃CH₂CH₂SH), 2-propanethiol (CH₃CHSHCH₃), hexanal (CHOCH₂CH₂CH₂CH₂CH₃), acetaldehyde (CH₃CHO), styrene (C₆H₅CHCH₂), heptanal (CHOCH₂CH₂CH₂CH₂CH₂CH₃), propyl benzene (C₆H₅CH₂CH₂CH₃), ethyl benzene (C₆H₅CH₂CH₃), phenol (C₆H₅OH), ethyl acetate (C₄H₈O₂), nonane (C₉H₂₀), 1-propanol (CH₃CH₂CH₂OH), 1,2,4-trimethyl benzene (C₆H₃(CH₃)₃), decane (CH₃CH₂CH₂CH₂CH₂CH₂CH₂CH₂CH₂CH₃), methyl amine (CH₃NH₂), melamine (C₃H₆N₆), dimethyl amine ((CH₃)₂NH), trimethyl amine ((CH₃)₃N) undecane (CH₃CH₂CH₂CH₂CH₂CH₂CH₂CH₂CH₂CH₂CH₃), isotopic CO₂ and CO in the presence of water. Of particular interest in the 5-12 um spectral ranges are molecules such as methane, ammonia, ethane, formaldehyde, nitric oxide, ethanol, ozone, acetone, carbonyl sulfide, sulfur dioxide, benzene, sulfur hexafluoride (SF₆), glucose (C₆H₁₂O₆), methanol, isobutene, isopropanol, dimethylsulfide, isoprene, pentane, carbon disulfide (CS₂), toluene, butane, 1-hexene, 2-hexene, 3-hexene, methyl cyclopentane, hexanal, styrene, heptanal, propylbenzene, 1,2,4-trimethyl benzene, decane, undecane, hydrogen peroxide (H₂O₂) and various polyaromatic hydrocarbons (PAH) such as anthracene (C₁₄H₁₀), benzo[a]pyrene (C₂₀H₁₂), chrysene (C₁₈H₁₂), coronene (C₂₄H₁₂), corannulene (C₂₀H₁₀), tetracene (C₁₈H₁₂), naphthalene (C₁₀H₈), pentacene (C₂₂H₁₄), phenanthrene (C₁₄H₁₀), pyrene (C₁₆H₁₀), triphenylene C₁₈H₁₂, and ovalene (C₃₂H₁₄). However, these molecules are only listed as examples; any molecules with absorption bands in the detection bandwidth can be detected.

For mass applications of trace gas detection systems, the frequency rulers as described here can further be substituted with other mid IR light sources, such as quantum cascade laser based frequency combs, micro-resonators, fiber or waveguide based supercontinuum sources or sources based on difference frequency generation (DFG). For example mid IR continuum light sources have been disclosed in U.S. patent application Ser. No. 13/458,058, ('058), entitled “Broadband generation of coherent continua with optical fibers”, filed Apr. 27, 2012. Mid IR sources based on DFG have been disclosed in the following U.S. patents and applications: U.S. patent application Ser. No. 13/232,470, ('470), entitled Optical parametric amplification, optical parametric generation, and optical pumping in optical fibers systems”, filed Sep. 14, 2011; U.S. patent application Ser. No. 13/682,309, ('309), entitled “A compact, coherent, high brightness light source for the mid-IR and Far-IR”, filed Nov. 20, 2012; and U.S. Pat. No. 8,237,122, entitled “Optical scanning and imaging systems based on dual pulsed laser systems”.

Thus, the invention has been described in several embodiments. It is to be understood that the embodiments are not mutually exclusive, and elements described in connection with one embodiment may be combined with, or eliminated from, other embodiments in suitable ways to accomplish desired design objectives.

In at least one embodiment the present invention features a trace gas detection system. The trace gas detection system includes an optical source producing as a primary output a frequency spectrum having a 1^{st} comb with a 1^{st} comb spacing within a 1^{st} spectral range. An enhancement cavity contains a sample gas for spectroscopic measurement. The enhancement cavity is configured to receive the primary output of the optical source and to produce a secondary output. The enhancement cavity is characterized by having a 2^{nd} comb of approximately equidistant spectral resonances and a 2^{nd} comb spacing within a 2^{nd} spectral range, wherein the 1^{st} spectral range and 2^{nd} spectral range overlap. The system includes a dither mechanism configured to modulate the relative position between the 1^{st} comb and the 2^{nd} comb at a dither frequency, f_{d}, and to impart variations of the relative position in optical frequency space larger than an optical linewidth of the cavity resonances. A feedback mechanism is included, and coupled to the dither mechanism to stabilize the location of the 1^{st} comb lines with respect to the resonances of the 2^{nd} comb on a time scale much greater than a dither period, T_{d}=1/f_{d}. The system further includes a Fourier transform spectrometer configured to receive the secondary output, and to measure the spectrum of a time-averaged signal transmitted by the cavity over a time scale much longer than T_{d}.

In any or all embodiments a first comb may be characterized by having a carrier envelope offset frequency, fₒ, and allowable variations thereof in the absence of phase locking of a carrier envelope offset frequency.

In any or all embodiments an enhancement cavity may have a comb spacing which is an integer fraction or integer multiple of a 1^{st} comb spacing.

In any or all embodiments a trace gas detection system includes optical source which may include a mode-locked laser, an OPO, OPA, DFG system, quantum cascade laser or micro-resonator.

In any or all embodiments a gas delivery system may be included to insert and optionally extract a gas sample into or from a cavity.

In any or all embodiments a trace gas detection system may be configured for detection of optical spectra at wavelengths>1600 nm.

In any or all embodiments a trace gas detection system may be configured for detection of optical spectra at wavelengths in the wavelength range from 3 to 6 μm.

In any or all embodiments a trace gas detection system may be configured for detection of optical spectra at wavelengths in the wavelength range from 5 to 15 μm.

In any or all embodiments a dither period, T_{d}, may be derived from the zero-crossings of an interference pattern generated by a reference laser within a Fourier transform spectrometer.

In any or all embodiments a Fourier transform spectrometer may be configured to sample a signal transmitted through a cavity in synchronism with zero-crossings of an interference pattern.

In any or all embodiments a feedback mechanism may be configured for detecting transmission peaks from an enhancement cavity and to provide electronic feedback to a cavity mirror so as to produce an approximately uniform time spacing of transmission peaks.

In any or all embodiments a dither mechanism may be configured to modulate the position of cavity spectral resonances of an enhancement cavity via movement of one of the cavity mirrors.

In any or all embodiments a dither mechanism may be configured to modulate the comb spacing of a 1^{st} comb.

In any or all embodiments dither mechanism may be configured to modulate a carrier envelope offset frequency of a 1^{st} comb.

In any or all embodiments an optical source may be diode pumped, and a carrier envelope offset frequency modulated by dithering diode power with a supplementary pump signal.

In any or all embodiments a carrier envelope offset frequency may be modulated with a graphene modulator.

In any or all embodiments an acousto-optic frequency shifter may be provided to modulate a carrier envelope offset frequency of a 1^{st} comb.

In any or all embodiments dither period, T_{d}, may be greater than about 100 μs, corresponding to a dither frequency less than about 10 kHz.

In any or all embodiments a dither period may be in the range from about 1 μsec to about 100 μs, corresponding to a dither frequency in the range from about 10 kHz to 1 MHz.

In any or all embodiments a dither mechanism may be configured to modulate the position of a 1^{st} or 2^{nd} frequency comb by about one free spectral range of an enhancement cavity.

In any or all embodiments a dither mechanism may be configured to modulate the position of the 1^{st} or 2^{nd} frequency comb by a fraction of a free spectral range of an enhancement cavity.

In any or all embodiments a dither mechanism may be configured to modulate a position of a 1^{st} or 2^{nd} frequency comb by more than a free spectral range of an enhancement cavity.

In any or all embodiments a Fourier transform spectrometer may be configured to sample more than two cavity transmission peaks between two zero-crossings.

In any or all embodiments a Fourier transform spectrometer may be configured to sample a uniform number of cavity transmission peaks between two zero-crossings.

In any or all embodiments a Fourier transform spectrometer may be configured to sample a signal transmitted through a cavity at time intervals much smaller than the time intervals between two adjacent zero crossings.

In any or all embodiments an optical source may be configured as a frequency comb source with repetition rate, fᵣₑₚ, and carrier envelope offset frequency, fₒ, phase locked to reference signals via phase locked loop(s).

In any or all embodiments feedback loops may be arranged in a feedback mechanism.

In any or all embodiments a trace gas detection system may be configured for breath analysis.

In any or all embodiments a trace gas detection system may be configured for detection of volatile organic compounds.

In any or all embodiments a trace gas detection system may be configured for detection of endogeneous compounds.

In any or all embodiments a trace gas detection system may be configured for cancer detection via breath analysis of volatile organic and/or endogenous compounds.

In at least one embodiment the present invention features a trace gas detection system. The trace gas system includes an optical source producing as a primary output a frequency spectrum having a 1^{st} comb with a 1^{st} comb spacing within a 1^{st} spectral range, the 1^{st} spectral range including wavelengths>1600 nm. An enhancement cavity contains a sample gas for spectroscopic measurement. The enhancement cavity is configured to receive the primary output of the optical source and to produce a secondary output. The enhancement cavity is characterized by having a 2^{nd} comb of approximately equidistant spectral resonances and a 2^{nd} comb spacing within a 2^{nd} spectral range. The 1^{st} spectral range and 2^{nd} spectral range overlap. A dither mechanism is included and is configured to modulate the relative position between the 1^{st} comb and the 2^{nd} comb at a dither frequency, f_{d}, and to impart variations of the relative position in optical frequency space larger than an optical linewidth of the cavity resonances. A feedback mechanism is coupled to the dither mechanism to stabilize the location of the 1^{st} comb lines with respect to the resonances of the 2^{nd} comb on a time scale much greater than a dither period, T_{d}=1/f_{d}. The trace gas detection system includes a spectroscopic measurement tool including an optical detection system. The tool is configured for frequency resolved detection of a time-averaged signal transmitted through the enhancement cavity.

In any or all embodiments an optical detection system may include a one dimensional detector array or a two dimensional detector array.

In at least one embodiment the present invention features a trace gas system. The system includes an optical source producing as a primary output a frequency spectrum having a 1^{st} comb with a 1^{st} comb spacing within a 1^{st} spectral range. An enhancement cavity contains a sample gas for spectroscopic measurement. The enhancement cavity is configured to receive the primary output of the optical source and to produce a secondary output. The enhancement cavity is characterized by having a 2^{nd} comb of approximately equidistant spectral resonances and a 2^{nd} comb spacing within a 2^{nd} spectral range. The 1^{st} spectral range and 2^{nd} spectral range overlap. The system includes a dither mechanism configured to modulate the relative position between the 1^{st} comb and the 2^{nd} comb at a dither frequency, f_{d}, and to impart variations of the relative position in optical frequency space larger than an optical linewidth of the cavity resonances. A spectroscopic measurement tool is included and configured to receive the secondary output, and to measure the spectrum of a time-averaged signal transmitted by the cavity over the time scale much longer than T_{d}=1/f_{d}. The spectroscopic tool is configured to provide a signal for synchronization of dithering with spectroscopic data acquisition.

In any or all embodiments a spectroscopic tool may include a Fourier transform spectrometer (FTS) having a reference laser from which an interference signal is generated, and the FTS may be configured to sample a signal transmitted through an enhancement cavity in synchronism with zero crossings of an interference signal.

In any or all embodiments the system may include a feedback mechanism coupled to a dither mechanism, wherein a dither period, T_{d}, is derived from zero crossings of an interference signal and used to control a dither mechanism via a feedback mechanism.

For purposes of summarizing the present invention, certain aspects, advantages and novel features of the present invention are described herein. It is to be understood, however, that not necessarily all such advantages may be achieved in accordance with any particular embodiment. Thus, the present invention may be embodied or carried out in a manner that achieves one or more advantages without necessarily achieving other advantages as may be taught or suggested herein.

The term “or” is used in this application its inclusive sense (and not in its exclusive sense), unless otherwise specified. In addition, the articles “a” and “an” as used in this application and the appended claims are to be construed to mean “one or more” or “at least one” unless specified otherwise

Thus, while only certain embodiments have been specifically described herein, it will be apparent that numerous modifications may be made thereto without departing from the spirit and scope of the invention. Further, acronyms are used merely to enhance the readability of the specification and claims. It should be noted that these acronyms are not intended to lessen the generality of the terms used and they should not be construed to restrict the scope of the claims to the embodiments described therein.

What is claimed is:

1. A trace gas detection system, comprising,
An optical source producing as a primary output a frequency spectrum comprising a 1^{st} comb with a 1^{st} comb spacing within a 1^{st} spectral range;
an enhancement cavity containing a sample gas for spectroscopic measurement, said enhancement cavity configured to receive the primary output of said optical source and to produce a secondary output, said enhancement cavity characterized by having a 2^{nd} comb of approximately equidistant spectral resonances and a 2^{nd} comb spacing within a 2^{nd} spectral range,
wherein said 1^{st} spectral range and 2^{nd} spectral range overlap;
a dither mechanism configured to modulate the relative position between said 1^{st} comb and said 2^{nd} comb at a dither frequency, f_{d}, and to impart variations of said relative position in optical frequency space larger than an optical linewidth of said cavity resonances;
a feedback mechanism coupled to said dither mechanism to stabilize the location of said 1^{st} comb lines with respect to the resonances of said 2^{nd} comb on a time scale much greater than a dither period, T_{d}=1/f_{d}, and
a Fourier transform spectrometer configured to receive said secondary output, and to measure the spectrum of a time-averaged signal transmitted by said cavity over said time scale much longer than T_{d}.

2. A trace gas detection system according to claim 1, wherein said first comb is characterized by having a carrier envelope offset frequency, fₒ, and allowable variations thereof of in the absence of phase locking of said carrier envelope offset frequency.

3. A trace gas detection system according to claim 1, said enhancement cavity having a comb spacing which is an integer fraction or integer multiple of said 1^{st} comb spacing.

4. A trace gas detection system according to claim 1, wherein said optical source comprises a mode-locked laser, an OPO, OPA, DFG system, quantum cascade laser or micro-resonator.

5. A trace gas detection system according to claim 1, further comprising a gas delivery system to insert and optionally extract a gas sample into or from said cavity.

6. A trace gas detection system according to claim 1, wherein said trace gas detection system is configured for detection of optical spectra at wavelengths>1600 nm.

7. A trace gas detection system according to claim 1, said trace gas detection system configured for detection of optical spectra at wavelengths in the wavelength range from 3-6 μm.

8. A trace gas detection system according to claim 1, said trace gas detection system configured for detection of optical spectra at wavelengths in the wavelength range from 5-15 μm.

9. A trace gas detection system according to claim 1, wherein said dither period, T_{d}, is derived from the zero-crossings of an interference pattern generated by a reference laser within said Fourier transform spectrometer.

10. A trace gas detection system according to claim 9, wherein said Fourier transform spectrometer is configured to sample the signal transmitted through the cavity in synchronism with said zero-crossings of said interference pattern.

11. A trace gas detection system according to claim 1, said feedback mechanism configured for detecting the transmission peaks from said enhancement cavity and providing electronic feedback to a cavity mirror to produce an approximately uniform time spacing of said transmission peaks.

12. A trace gas detection system according to claim 1, wherein said dither mechanism is configured to modulate the position of the cavity spectral resonances of said enhancement cavity via movement of one of the cavity mirrors.

13. A trace gas detection system according to claim 1, wherein said dither mechanism is configured to modulate the comb spacing of said 1^{st} comb.

14. A trace gas detection system according to claim 1, wherein said dither mechanism is configured to modulate the carrier envelope offset frequency of said 1^{st} comb.

15. A trace gas detection system according to claim 14, wherein said optical source is diode pumped, and said carrier envelope offset frequency is modulated by dithering the diode power with a supplementary pump signal.

16. A trace gas detection system according to claim 14, wherein said carrier envelope offset frequency is modulated with a graphene modulator.

17. A trace gas detection system according to claim 1, further comprising an acousto-optic frequency shifter to modulate the carrier envelope offset frequency of said 1^{st} comb.

18. A trace gas detection system according to claim 1, wherein said dither period, T_{d}, is greater than about 100 μs, corresponding to a dither frequency less than about 10 kHz.

19. A trace gas detection system according to claim 1, wherein said dither period is in the range from about 1 μsec to about 100 μs, corresponding to a dither frequency in the range from about 10 kHz to 1 MHz.

20. A trace gas detection system according to claim 1, said dither mechanism configured to modulate the position of said 1^{st} or 2^{nd} frequency comb by about one free spectral range of said enhancement cavity.

21. A trace gas detection system according to claim 1, wherein said dither mechanism is configured to modulate the position of said 1^{st} or 2^{nd} frequency comb by a fraction of said free spectral range of said enhancement cavity.

22. A trace gas detection system according to claim 1, wherein said dither mechanism is configured to modulate the position of said 1^{st} or 2nd frequency comb by more than a free spectral range of said enhancement cavity.

23. A trace gas detection system according to claim 1, wherein said Fourier transform spectrometer is configured to sample more than two cavity transmission peaks between two zero-crossings.

24. A trace gas detection system according to claim 1, wherein said Fourier transform spectrometer is configured to sample a uniform number of cavity transmission peaks between two zero-crossings.

25. A trace gas detection system according to claim 1, said Fourier transform spectrometer configured to sample the signal transmitted through the cavity at time intervals much smaller than the time intervals between two adjacent zero crossings.

26. A trace gas detection system according to claim 1, wherein said optical source is configured as a frequency comb source with repetition rate, fᵣₑₚ, and carrier envelope offset frequency, fₒ, phase locked to reference signals via phase locked loops.

27. A trace gas system according to claim 26, wherein said feedback loops are arranged in said feedback mechanism.

28. A trace gas detection system according to claim 1, wherein said system is configured for breath analysis.

29. A trace gas detection system according to claim 1, wherein said system is configured for detection of volatile organic compounds.

30. A trace gas detection system according to claim 1, wherein said system is configured for detection of endogeneous compounds.

31. A trace gas detection system according to claim 1, wherein said system is configured for cancer detection via breath analysis of volatile organic and/or endogenous compounds.

32. A trace gas detection system, comprising,
An optical source producing as a primary output a frequency spectrum comprising a 1^{st} comb with a 1^{st} comb spacing within a 1^{st} spectral range, said first spectral range comprising wavelengths>1600 nm;
an enhancement cavity containing a sample gas for spectroscopic measurement, said enhancement cavity configured to receive the primary output of said optical source and to produce a secondary output, said enhancement cavity characterized by having a 2^{nd} comb of approximately equidistant spectral resonances and a 2^{nd} comb spacing within a 2^{nd} spectral range,
wherein said 1^{st} spectral range and 2^{nd} spectral range overlap;
a dither mechanism configured to modulate the relative position between said 1^{st} comb and said 2^{nd} comb at a dither frequency, f_{d}, and to impart variations of said relative position in optical frequency space larger than an optical linewidth of said cavity resonances;
a feedback mechanism coupled to said dither mechanism to stabilize the location of said 1^{st} comb lines with respect to the resonances of said 2^{nd} comb on a time scale much greater than a dither period, T_{d}=1/f_{d}, and
a spectroscopic measurement tool comprising an optical detection system, said tool configured for frequency resolved detection of a time-averaged signal transmitted through the enhancement cavity.

33. A trace gas detection system according to claim 32, wherein said optical detection system comprises a one dimensional detector array or a two dimensional detector array.

34. A trace gas system, comprising:
an optical source producing as a primary output a frequency spectrum comprising a 1^{st} comb with a 1^{st} comb spacing within a 1^{st} spectral range;
an enhancement cavity containing a sample gas for spectroscopic measurement, said enhancement cavity configured to receive the primary output of said optical source and to produce a secondary output, said enhancement cavity characterized by having a 2^{nd} comb of approximately equidistant spectral resonances and a 2^{nd} comb spacing within a 2^{nd} spectral range,
wherein said 1^{st} spectral range and 2^{nd} spectral range overlap;
a dither mechanism configured to modulate the relative position between said 1^{st} comb and said 2^{nd} comb at a dither frequency, f_{d}, and to impart variations of said relative position in optical frequency space larger than an optical linewidth of said cavity resonances; and
a spectroscopic measurement tool configured to receive said secondary output, and to measure the spectrum of a time-averaged signal transmitted by said cavity over said time scale much longer than T_{d}=1/f_{d},
wherein the spectroscopic tool is configured to provide a signal for synchronization of dithering with spectroscopic data acquisition.

35. A trace gas system according to claim 34, wherein said spectroscopic tool comprises a Fourier transform spectrometer (FTS) having a reference laser from which an interference signal is generated, and said FTS is configured to sample a signal transmitted through said enhancement cavity in synchronism with zero crossings of said interference signal.

36. A trace gas system according to claim 35, further comprising a feedback mechanism coupled to said dither mechanism, wherein a dither period, T_{d}, is derived from zero crossings of said interference signal and used to control said dither mechanism via said feedback mechanism.
